(19) United States
(12) Patent Application Publication
(10) Pub. No.: US 2012/0232621 A1
(43) Pub. Date: Sep. 13, 2012

(54) THERMAL TREATMENT DEVICE WITH VARIABLE HEAT DISTRIBUTION

(76) Inventors: Leo B. Kriksunov, Ithaca, NY (US); David W. Wynn, Huntington Valley, PA (US)

(21) Appl. No.: 13/414,006

(22) Filed: Mar. 7, 2012

Related U.S. Application Data
(60) Provisional application No. 61/451,312, filed on Mar. 10, 2011.

(51) Int. Cl. A61F 7/08

(52) U.S. Cl. 607/114; 607/96

(57) ABSTRACT

A thermal treatment device having variable heat distribution along the device is disclosed.

This application claims the benefit of U.S. provisional application 61/451,312 filed Mar. 10, 2011, the complete disclosure of which is hereby incorporated herein by reference for all purposes.

### FIELD OF THE INVENTION

The present invention is directed to a thermal treatment device. More particularly, the present invention relates to a thermal treatment device that has variable heat distribution along the device.

### BACKGROUND OF THE INVENTION

For users with aching muscles and sore joints, the application of heat can loosen stiff muscles, improve blood flow to the affected area, facilitate tissue repair, and create a feeling of relaxation. The application of heat to the skin has historically been used for pain relief of muscles and joints, as well as for the treatment of certain inflammatory conditions. The application of cold materials to the skin has also been used for similar treatments, especially for treating inflammatory responses such as joint inflammation.

Chemical formulations, such as iron powder formulations, that oxidize when exposed to air have been employed to generate heat. Electrical heating elements attached to a power source have also been employed.

U.S. Pat. No. 6,074,413 to Proctor & Gamble discloses a disposable thermal back wrap that contains one or more thermal packs.

U.S. Pat. No. 5,484,366 to Wilkinson discloses a belt that contains at least one packet to hold chemical gel packs. The gel packs may be heated or cooled to the desired temperature.

U.S. Pat. No. 5,605,144 discloses a garment that has at least one pouch for holding an air activated heat producing packet.

U.S. Application No. 20080200971 to Mycoal Products Corporation discloses a heating element that contains three or more segmented heating parts that contain an exothermic composition capable of heat generation upon contact with air that consists of a low temperature segment, an intermediate temperature segment and a high temperature segment.

U.S. Application No. 20080140165 to Kimberly-Clark Worldwide, Inc. discloses an article for delivering a heating sensation that includes a skin facing surface, a plurality of warm potions disposed on the skin facing surface and a plurality of cool portions disposed on the skin facing surface, wherein the warm potions and the cool portions are disposed in an alternating pattern.

There is a need in the art for improved thermal treatment devices.

### SUMMARY OF THE INVENTION

Currently marketed air-activated heat packs provide substantially uniform heat. Providing non-uniform heat distribution may result in better treatment outcomes and decreased side effects. Specifically, sensitization of the user's skin to the sensations of heat can be decreased when the skin is exposed to a heat source of spatially variable temperature. Additionally, higher temperature generating segments might operate for shorter time but provide for a very fast ramp-up of temperature, thus initiating treatment faster, while lower temperature segments might operate for a longer time but provide for a longer ramp-up of temperature, thus initiating treatment slower, but providing for a longer treatment. Thus, it is beneficial to have a non-uniform heat distribution in a heat pack for therapeutic applications. Further, the thermal grill effect, described, for example, in the aforementioned U.S. Application No. 20080140165 to Kimberly-Clark Worldwide, provides for a perceived sensation of heat without exposing the skin to potentially detrimental high temperatures.

U.S. Application No. 20080200971 to Mycoal Products Corporation (FIGS. 1, 2, 4, 5 and 7) and U.S. Application No. 20080140165 to Kimberly-Clark (FIGS. 1, 2 and 3) show thermal grill-like structures with multiple narrow sections. Manufacturing of such structures and particularly filling such structures with different heating mixtures and then sealing such structures is complex and requires highly specialized fill and seal equipment. Furthermore, the total quantity of the heating material in each heat pack is limited because of the presence of so many sealing areas around each narrow heating element filled with the heating mixture, resulting in low overall amount of heating mixture.

It is an object of the invention to provide a thermal treatment device that comprises variable heat distribution. The thermal treatment device is convenient in that different compositions may be placed into different portions of the thermal treatment device, resulting in a device which provides a hotter portion for a brief burst of time and a warmer portion for a longer portion of time. Typically, air activated heating device compositions are limited, in that the more rapidly the composition oxidizes upon exposure to air, the more rapidly it is extinguished. If a composition is extinguished too rapidly, it cannot provide long-term pain relief. In previous thermal treatment devices, manufacturers have had to compromise between the intensity of the heat and the duration of the heat delivery. It is advantageous to the user to have a device that provides an intense heating portion at the beginning of use, followed by a longer warming device. This can be provided using the device of the present invention.

### BRIEF DESCRIPTION OF THE FIGURES

FIGS. 1A, 1B and 1C-1 to 1C-6 depict embodiments of a thermal treatment device of the present invention, wherein (a) the device has a hot center and a warm periphery (FIG. 1A); (b) the device has a warm center and a hot periphery (FIG. 1B); and (c) the device has a non-heated center and a hot periphery (FIG. 1C-1 to 1C-6).

FIGS. 2A-1 to 2A-2 and 2B depict another embodiment of a thermal treatment device of the present invention, wherein (a) the device has a hot center with apertures and a warm periphery (FIGS. 2A-1 to 2A-2); and (b) the device has a warm center and a hot periphery with apertures (FIG. 2B).

FIGS. 3A, 3B, and 3C-1 to 3C-2 depict yet another embodiment of a thermal treatment device of the present invention, wherein (a) the device has a non-insulated hot center and an insulated warm periphery (FIG. 3A); (b) the device has an insulated warm center and a non-insulated hot periphery (FIG. 3B); and (c) the device has concentric insulation around a hot center (FIGS. 3C-1 to 3C-).

FIGS. 4A and 4B depict still yet another embodiment of a thermal treatment device of the present invention, wherein (a) the device has a hot center and a warm periphery prepared by placing a hot sub-pack on top of a warm sub-pack (FIG. 4A); and (b) the device has a warm center and a hot periphery prepared by placing a warm sub-pack between two hot sub-packs (FIG. 4B). In one embodiment, a sub-pack may be defined as at least one portion of the total thermal treatment device, wherein such sub-pack is enclosed with a separate portion of the heating composition. The sub-pack is smaller in dimension than the total thermal treatment device, which may comprise one or more sub-packs.

FIGS. 5A and 5B depict still yet another embodiment of a thermal treatment device of the present invention, wherein (a) the device has a hot center and a warm periphery, wherein two or more compartments are filled with different air-activated heat mixtures; and (b) the device has a hot center and a warm periphery, wherein three or more compartments are filled with different air-activated heat mixtures.

FIG. 6A is a top view of an example of a thermal treatment device of the invention.

FIG. 6B is a side view of the thermal treatment device of FIG. 6A.

### DETAILED DESCRIPTION OF THE INVENTION

The following detailed description references the accompanying drawings that show some example aspects of the disclosure. These example aspects are described in sufficient detail to enable those skilled in the art to practice the disclosure. It is to be understood that other aspects may be utilized, or structural changes made, such that the detailed description should not be considered as limiting the scope of the claims.

As used herein, the term “hot” or “hot portion” and its plural, and the term “warm” or ^{“}warm portion” and its plural, refer to the portion(s) of the device that are exothermic or potentially exothermic. As used herein, exothermic refers to emitting heat and such heat can be created by a chemical reaction, by electrically-resistive heating, by warmed fluid, or by any other suitable means. The “hot portion” and/or the “warm portion” may actually feel hot and/or warm, respectively, as it would upon activation, or the “hot portion” and/or the “warm portion” may be potentially hot or heatable to a hot temperature and/or warm or warmable as they would be before activation in that it includes material that will give off heat upon activation.

“Hot” refers to temperatures 5° C. and more hotter than skin temperature, i.e., about 5-20° C. hotter than normal skin temperature of 36.6° C. “Warm” refers to skin temperature and up to 5° C. higher. Further, a portion of the device can be not heated at all, in which case the temperature of that area will be at the skin temperature or slightly above, i.e., “warm”.

The hot and warm portions may be a variety of sizes, designs, configurations, shapes, temperatures, and orientations as long as the thermal treatment device generates a perception of heat without physiologically damaging the individual when the individual uses the thermal treatment device. The relative size and shape of the hot and warm portions that form the thermal treatment device will depend on the application and where the thermal treatment device is used.

The thermal treatment device can be employed, e.g., as a heating pad, a heat therapy device, a patch, a band or a pouch.

The present invention relates to a thermal treatment device for managing muscle and joint pain that is worn in close proximity to the skin of an individual. The thermal treatment device offers the benefit of improved therapeutic relief in one embodiment, by providing an interval of more intense heat for a shorter period of time, followed by a longer interval of less intense heat. In one embodiment, the hot portion may last for up to about 30 minutes, e.g. up to about 1 hour, e.g., up to about 2 hours; and the warm portion may last for up to about 6 hours, e.g., up to about 8 hours, e.g., up to about 12 hours, e.g., up to about 24 hours. This allows for a user to experience an initial period of relief of pain through a more intense heat application, followed by a longer warm period of heat. The initial period also serves as an indicator to the user that the device is working as intended. In one embodiment, the hot portion or feeling of heat can indicate to the user that the thermal device has been sufficiently exposed to air upon opening that it may be placed under clothing, where air exposure is more limited, solving the issue with current thermal treatment devices where a user has to wait for it to heat up. The device of the present invention also provides for a device that can deliver two temperatures to the pain and muscle receptors over time, since such receptors typically become acclimated or accustomed to one temperature.

Based on the analysis of the user perception and actual temperature measurements on the user's body, providing non-uniform heat, such as when hot zones are intermittent with warm and/or non-heated zones should result in perception of a hotter heat pack, decreased side effects and better treatment outcome. Advantageously, the variable heat applied to the body of the user will result in less sensitization of the skin and better breath-ability of the skin. Advantageously, the presence of a warm region and/or a non-heated cutout zone in the vicinity of the heated zone will result in a sensory perception of hotter heat pack at the same or lower effective temperature, thus decreasing the probability of overheating the skin. Advantageously, heat packs having lower temperature will be perceived as hotter by the user, thus decreasing the probability of undesirable side effects associated with very hot heat packs.

The thermal treatment device includes an outer surface comprising a skin facing surface, wherein the skin facing surface comprises variable heat distribution.

The thermal treatment device of the present invention will typically be worn in contact with the skin, either on top of a garment or beneath a garment. In one embodiment, the thermal treatment device includes an adhesive, which may adhere to a garment or to the skin.

The thermal treatment device may be constructed from a disposable, breathable, non-woven fabric. In one embodiment, the device is configured to have an air permeable front material connected to an adhesive backing. In another embodiment, an air permeable front material and adhesive backing enclose and contain an air activated exothermic reaction mixture.

In one embodiment, the thermal treatment device of the present invention comprises a thermally active component that delivers heat for therapeutic purposes.

In another embodiment, the thermal treatment device includes a thermal reservoir comprising a thermal composition.

The thermal composition can be any suitable material for either generating or holding heat. In one embodiment, the thermal composition emits heat from about 1 to about 10° C. above the skin surface temperature of an individual.

In one embodiment, a temperature sensor, such as a thermocouple, placed directly on the skin of the user and then covered by a thermal treatment device of the present invention, i.e., a temperature sensor positioned between the body of the user and the thermal treatment device can be used to measure and adjust the temperature so that it is above the normal body temperature of about 36-37° C. but below the temperatures which can be detrimental after a prolonged exposure, such as above 50-60° C. In one embodiment, the temperature measured as described above is from about 38° C. to about 50° C., such as from about 39° C. to about 45° C.

In one particular embodiment, the thermal reservoir comprises thermal fill materials that are a mixture of substances that react exothermically. For example, several commercial hand warmers and therapeutic heat products contain an iron powder based mixture that liberates heat as the iron is oxidized upon exposure to air. These types of systems are described in detail, for example, in U.S. Pat. No. 5,918,590 to Proctor & Gamble. It is known in the art to formulate these mixtures to maintain a temperature of at least about 40° C. for at least about 4 hours, and up to about 24 hours. Depending on the application or desired product design, the temperature may be maintained for at least about 8 hours, at least about 10 hours, at least about 12 hours, at least about 16 hours, or at least about 24 hours, with varying degrees of rate of heating up.

In certain embodiments, the temperature between an individual's skin and the thermal treatment device when measured by a thermocouple positioned between skin and the thermal treatment device is about 38° C., about 40° C., about 41° C., about 45° C., or about 50° C.

In one embodiment, the thermal reservoir may include an enclosure. The enclosure can be any material that surrounds the thermal reservoir or the thermal composition within the thermal reservoir. In one embodiment, the enclosure is a pouch constructed of breathable non-woven fabric. In yet another embodiment, the enclosure is constructed from a woven textile fabric.

In certain embodiments, the thermal treatment device can be substantially flat having a rectangular or elliptical or oval or other geometrical shape, with the thickness of the device ranging from about 1 millimeters to about 30 millimeters, and the other dimensions of the device adapted to fit into an outline frame having a rectangular shape and dimensions of from about 60×80 mm to about 200×500 mm. In one embodiment the thermal treatment device has a thickness of about 3 to about 8 mm and is a rectangle 80 mm×120 mm; in another embodiment the thermal treatment device is a rectangle 100 mm×150 mm; in another embodiment the thermal treatment device is a rectangle 150 mm×250 mm. In another embodiment the thermal treatment device is an ellipse having gross dimensions of 100 mm×150 mm.

The thermal treatment device may be worn in close proximity to an individual's skin or may be directly applied to the skin. In one embodiment, the thermal treatment device may be applied to clothing where the heat is transferred through the clothing to the skin. When direct application is employed, an adhesive may be used. Any suitable adhesive that is safe and effectively enables the thermal treatment device to adhere to the skin may be used. Suitable adhesives include, for example, hydrogels, silicone adhesives, hot melt adhesives, and the like. Ideally, the adhesive should permit the thermal treatment device to be applied and conform to the skin contact surface area. In some cases, the adhesive may facilitate the even distribution of heat or warmth across the skin area covered by the thermal treatment device. The thermal treatment device can have an adhesive layer on the side facing the skin, said layer preferably is a continuous layer covering substantially all of the device; in one embodiment the adhesive is an intermittent layer exemplified by a plurality of adhesive stripes. In one embodiment the adhesive is a hydrogel-based adhesive, which supplies “moist” heat. In another embodiment the adhesive is a non-hydrogel adhesive.

The thermal treatment device may be adapted to be inexpensive, light-weight entirely disposable and easily portable, thus allowing travel and mobility.

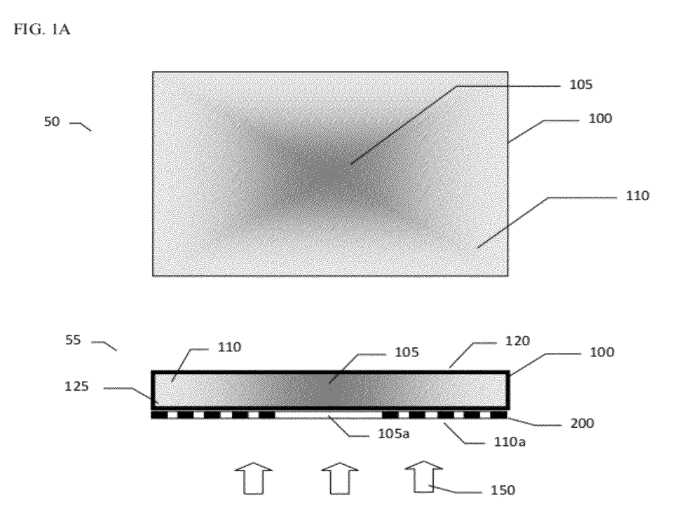

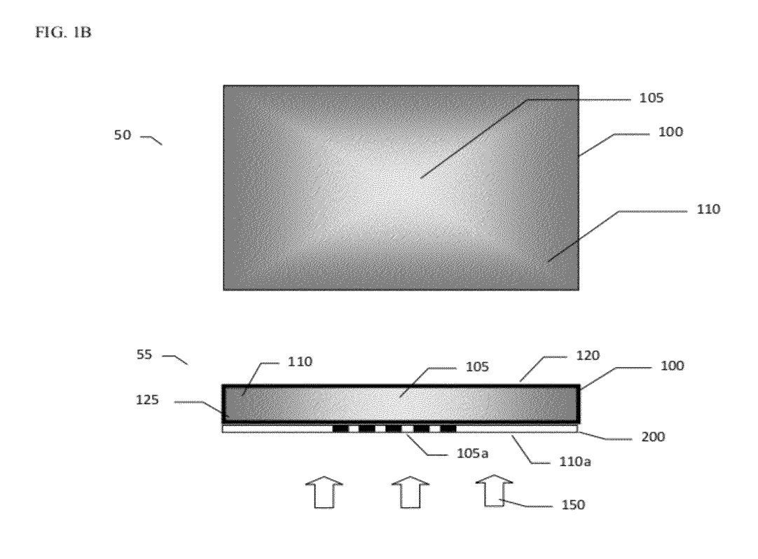

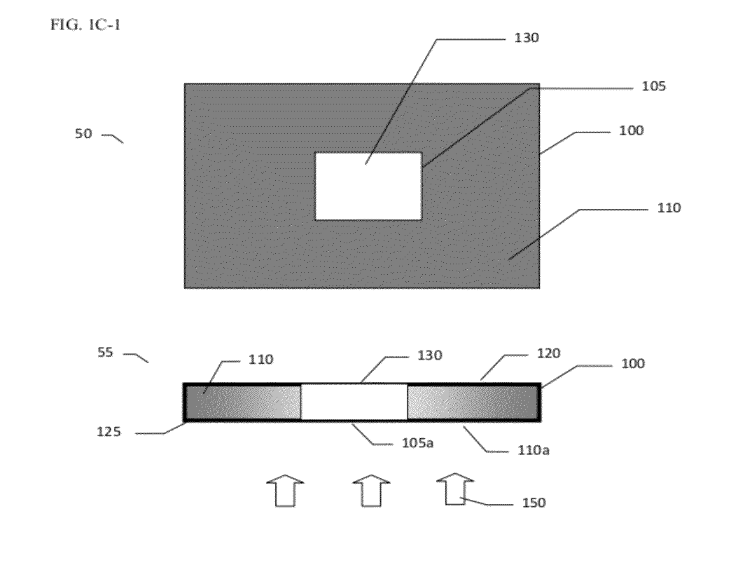

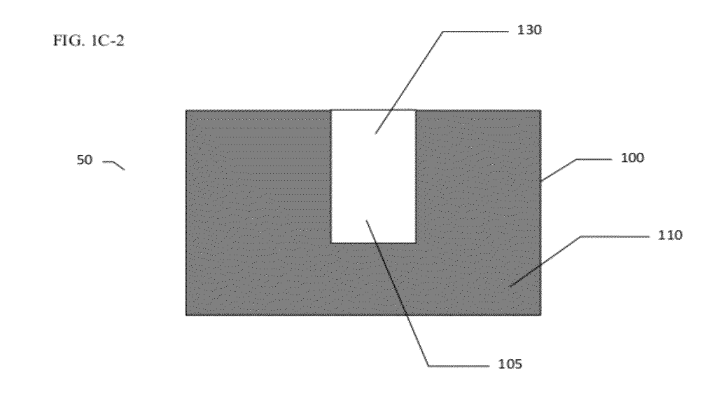

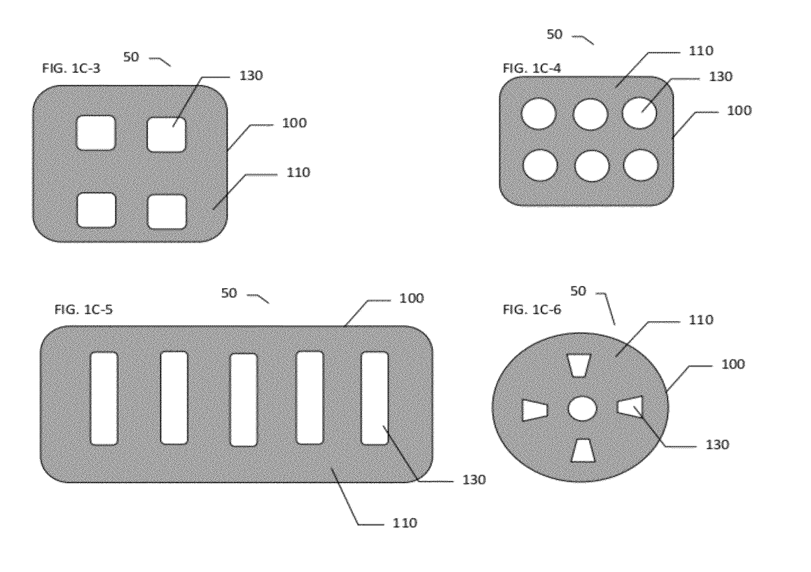

FIG. 1A is a schematic representation of an embodiment of the invention, showing top (body-facing) view 50 and side view 55 of a heat pack 100 having a shape of a substantially flat rectangle. The heat pack 100 has a hotter center 105 and a warm and/or cooler periphery 110 as further illustrated by the denser shadowing in the center 105 of heat pack 100 vs. periphery 110. Body facing side 120 has a different construction vs. air-intake side 125, with air intake side of the envelope being microporous to allow ingress of air as shown by arrows 150 into the heat-generating mixture (not shown) contained within the envelope of the heat pack. The envelope pores are typically from about 0.1 micron to about 25 microns.

The embodiment of the thermal treatment device of the present invention has a warmer center 105. In the embodiment shown in FIG. 1A, an additional air-permeability modifying member 200 is attached to air-intake side 125, leaving air permeability intact in the center 105 but restricting air permeability in the periphery 110. The air-permeability modifying member 200 is a layer of fabric or polymer having a variable permeability with high permeability or a cut-out in the center 105a and lower permeability on the periphery 110a as illustrated by shadowed regions on the periphery 110a of the air-permeability modifying member 200. Restricting the flow of air to the periphery 110 of heat pack 100 results in warm and/or cooler periphery 110 and hotter center 105. In one embodiment (not shown), air-permeability modifying member 200 is a microporous sticker of the same dimensions as the dimensions of the heat pack 100, said sticker placed onto air-intake side 125 and said sticker having a cutout in the center of the sticker.

In one embodiment, the air-permeability modifying member 200 is an air-permeable fabric attached to air-intake side 125 with an adhesive which selectively blocks some of the micropores, leaving air permeability intact in the center 105 but restricting air permeability on the periphery 110. In this embodiment, more adhesive or adhesive stripes are positioned on the periphery 110 and less adhesive or no adhesive is positioned in the center 105.

Referring now to FIG. 1B a schematic representation of an embodiment of the invention is shown with heat pack 100 having a warm and/or cooler center 105 and a hotter periphery 110 as further illustrated by the denser shadowing in the periphery 110 of heat pack 100 vs. center 105. Air-permeability modifying member 200 is attached to air-intake side 125, leaving air permeability intact in the periphery 110 but restricting air permeability in the center 105. In one embodiment (not shown), air-permeability modifying member 200 is a microporous sticker placed onto the center 105 on air-intake side 125, said sticker having dimensions smaller than the dimensions of the heat pack 100.

In one embodiment the air-permeability modifying member 200 is an air-permeable fabric attached to air-intake side 125 with an adhesive which selectively blocks some of the micropores, leaving air permeability intact on the periphery 110 but restricting air permeability in the center 105. In this embodiment, more adhesive or adhesive stripes are positioned in the center 105 and less adhesive or no adhesive is positioned on the periphery 110.

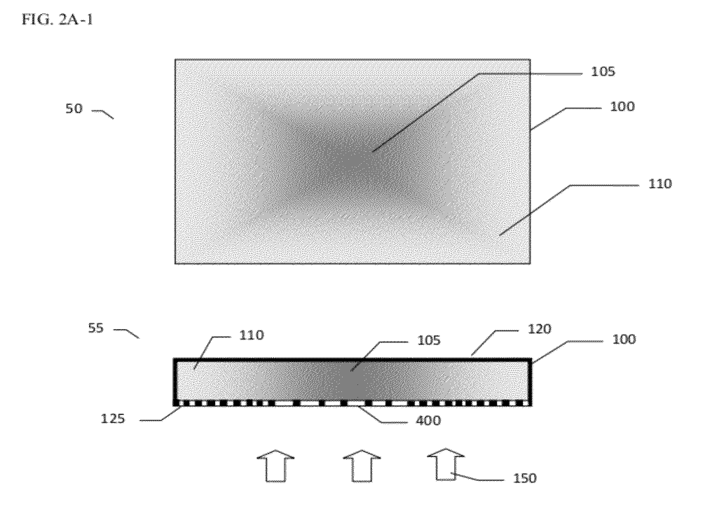

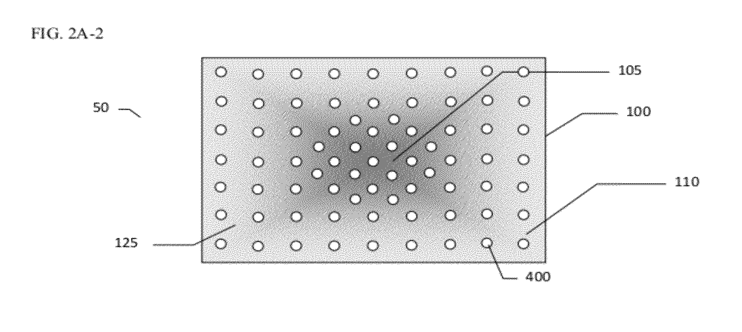

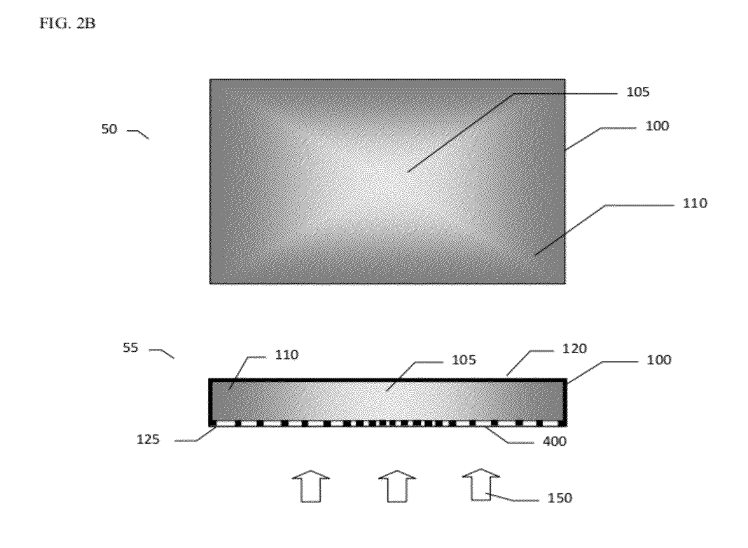

Referring now to FIG. 1C-1, the heat pack 100 has at least one cutout 130. In the embodiment shown, the cutout is substantially in the center 105 and has dimensions of at least 10% of the area of the heat pack 100 as seen on the top (body-facing) view 50, more preferably, 20%-50% of the area of the heat pack 100. The cutout can be rectangular, oval, polygonal, elliptical, triangular, or any suitable shape. In one embodiment the inside edges of the cutout are sealed. In one embodiment, the cutout is not contained within the perimeter of the heatpack, as illustrated in FIG. 1C-2. The presence of the cutout 130 advantageously provides for a warm and/or cooler zone within the treated area and also provides for an area wherein the user's skin can more easily evaporate moisture.

Referring now to FIGS. 1C-3 through 1C-6, several additional embodiments having multiple cut-outs are shown, wherein the elements listed are the same elements employed in FIG. 1C-1. As discussed above, based on user perception, providing non-uniform heat, such as when hot zones are intermittent with warm and/or non-heated zones should result in better treatment outcome, perception of a hotter heat pack, and decreased side effects. Advantageously, the variable heat applied to the body of the user will result in less sensitization of the skin and better breath-ability of the skin. Advantageously, the presence of a warm and/or non-heated cutout zone in the vicinity of the heated zone will result in a sensory perception of hotter adhesive heat pack at the same or lower effective temperature, thus decreasing the probability of overheating the skin. Advantageously, the cutout zones enable the evaporative cooling of skin through perspiration and further increase in the temperature difference between the heated zone and warm and/or non-heated cutout zone. Advantageously, heat pack having lower temperature will be perceived as hotter by the user thus decreasing the probability of undesirable side effects associated with very hot heat packs.

The packs and the cutouts can be rectangular, circular, polygonal, or of any geometric shape suitable for placing on the body or around the body. The packs can be worn on the body via attachment of the side coated with the adhesive to the skin. The cutouts have an area from about 0.5 cm² to about 5 cm² each. The number of cutouts in each heat pack is from one cutout to about 10 cutouts.

Referring now to FIG. 2A-1, representing an embodiment of the current invention, and showing top (body-facing) view 50 and side view 55 of a heat pack 100 having a shape of a substantially flat rectangle. The heat pack 100 has a hotter center 105 and a warm and/or cooler periphery 110 as further illustrated by the denser shadowing in the center 105 of heat pack 100 vs. periphery 110. Body facing side 120 has generally different construction vs. air-intake side 125, with air intake side being microporous to allow ingress of air as shown by arrows 150 into the heat-generating mixture (not shown) contained within the envelope of the heat pack.

The apertures 400 within the air-intake side 125 have higher density or larger diameter or both in the center of heat pack 100 thus rendering the center more air permeable. As a result center 105 is hotter vs. periphery 110. In one version of this embodiment the apertures are on present on one face of the heat pack, and are not representative of a cutout.

Referring now to FIG. 2A-2, the view from the air-intake side 125 is shown, schematically illustrating higher density apertures in the center 105 vs. periphery 110 resulting in hotter center 105. In an alternative embodiment (not shown) the apertures 400 in the center do not have higher density, but have a larger diameter.

Referring now to FIG. 2B, representing an embodiment of the invention, and showing top (body-facing) view 50 and side view 55 of a heat pack 100 having a shape of a substantially flat rectangle. The heat pack 100 has a colder center 105 and a hotter periphery 110 as further illustrated by the denser shadowing in periphery 110 of heat pack 100 vs. the center 105. The apertures 400 within the air-intake side of the envelope 125 have higher density or larger diameter or both on the periphery of heat pack 100 thus rendering the periphery more air permeable. As a result center 105 is colder vs. periphery 110.

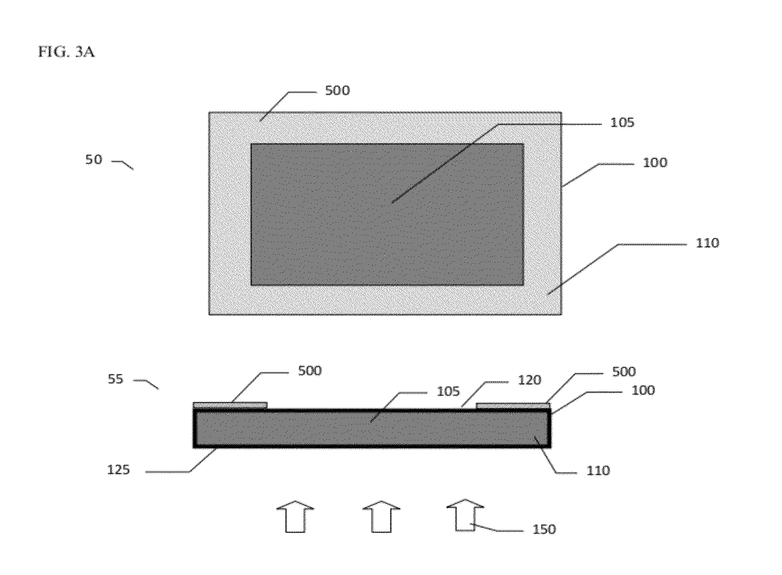

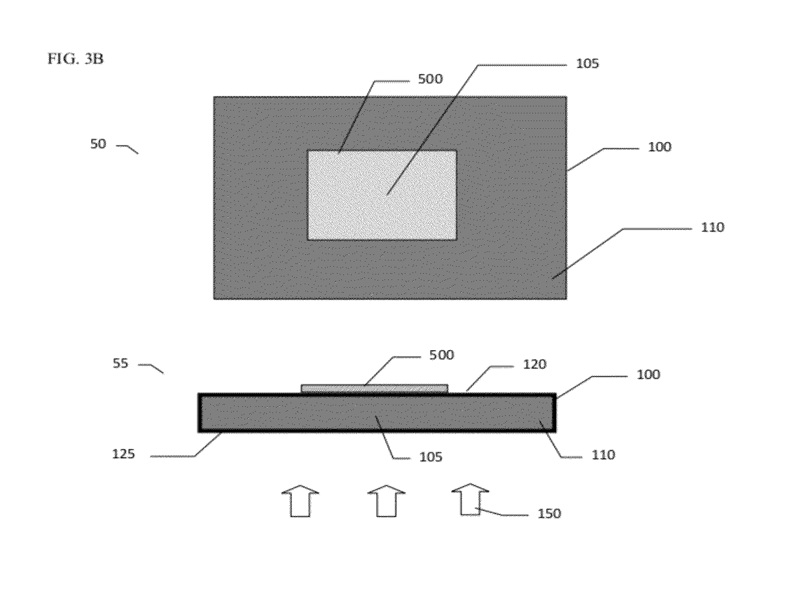

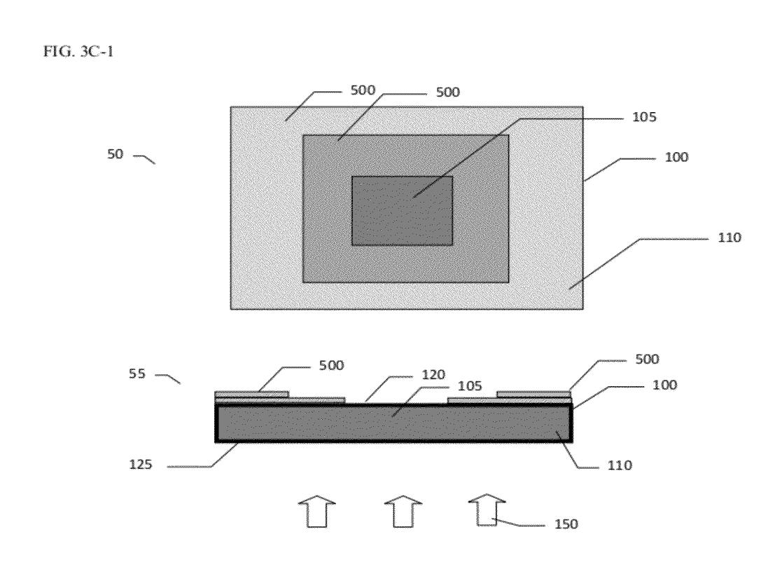

Referring now to FIG. 3A, representing an embodiment of the current invention, and showing top (body-facing) view 50 and side view 55 of a heat pack 100 having a shape of a substantially flat rectangle. The heat pack 100 is perceived by the user as having a hotter center 105 and a warm and/or cooler periphery 110 as further illustrated by the denser shadowing in the center 105 of heat pack 100 vs. periphery 110. Body facing side 120 has an insulating layer 500 disposed on the periphery 110 of heat pack 100 resulting in the perception that periphery 110 is colder.

Referring now to FIG. 3B, representing an embodiment of the current invention, and showing top (body-facing) view 50 and side view 55 of a heat pack 100 having a shape of a substantially flat rectangle. The heat pack 100 is perceived by the user as having a colder center 105 and a hotter periphery 110 as further illustrated by the denser shadowing in the periphery 110 of heat pack 100 on the top view 50. Body facing side 120 has an insulating layer 500 disposed only in the center 105 of heat pack 100 resulting in the perception that periphery 110 is hotter.

Referring now to FIG. 3C-1, representing an embodiment of the current invention, and showing top (body-facing) view 50 and side view 55 of a heat pack 100 having a shape of a substantially flat rectangle. The heat pack 100 has several concentric layers of insulating layers 500 on the body facing side 120 resulting in the heat pack being perceived by the user as having hotter center 105 where there is no insulation and then progressively colder periphery 110.

In the embodiment shown in FIG. 3C-2, stripes of insulating material 500 are applied intermittently to the body facing side 120 resulting in the heat pack being perceived by the user as having intermittent regions of hotter and warm and/or cooler zones.

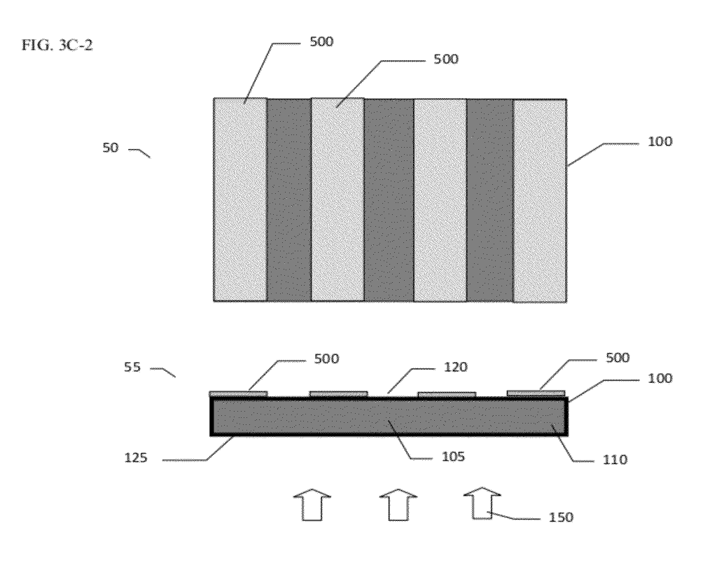

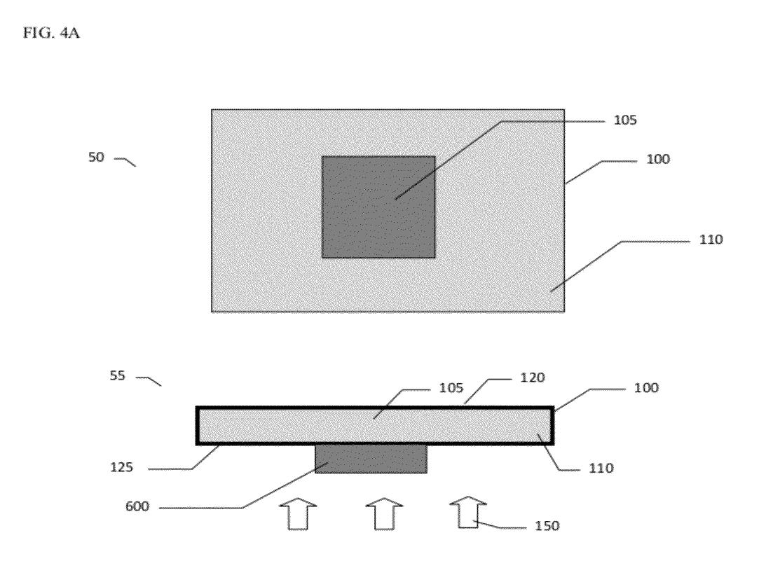

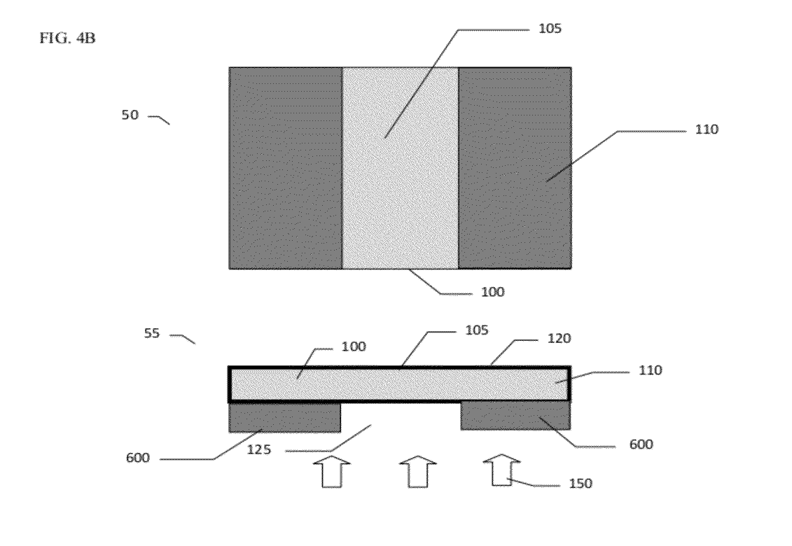

Referring now to FIG. 4A, the thermal treatment device has a hotter center 105 and a colder periphery 110 as further illustrated by the denser shadowing in the center 105 vs. periphery 110. In the embodiment shown in FIG. 4A, a hotter heat pack or subpack 600 of generally smaller dimensions vs. the main heat pack or subpack 100 is positioned in the center of the heat pack 100 on the air-intake side 125. The heat from the hotter heat pack 600 is then conducted through the main heat pack 100 towards the body facing side 120 resulting in the thermal treatment device with hotter center 105.Referring now to FIG. 4B, the thermal treatment device has a warm and/or cooler center 105 and a hotter periphery 110 as further illustrated by the denser shadowing of the periphery 110 on top (body-facing side) view 50. In the embodiment shown, two hotter heat packs or subpacks 600 are positioned on two sides of the main heat pack 100, on the air-intake side 125. The heat from the hotter heat packs 600 is then conducted through the main heat pack 100 towards the body facing side 120 resulting in the thermal treatment device with hotter periphery 110.

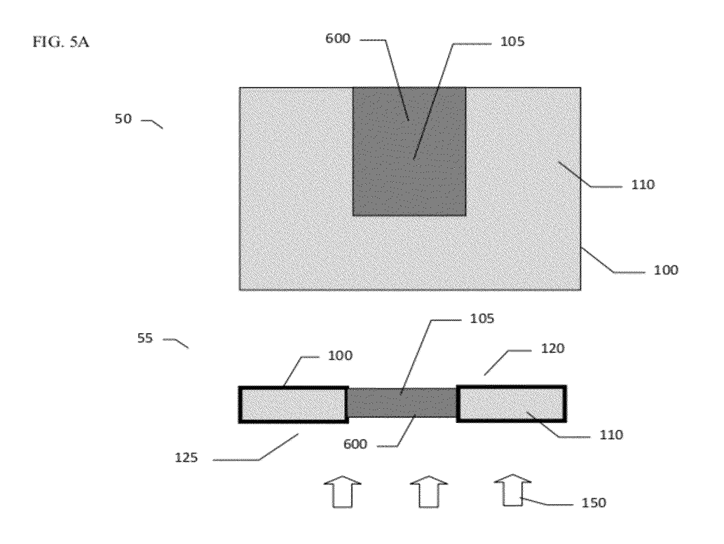

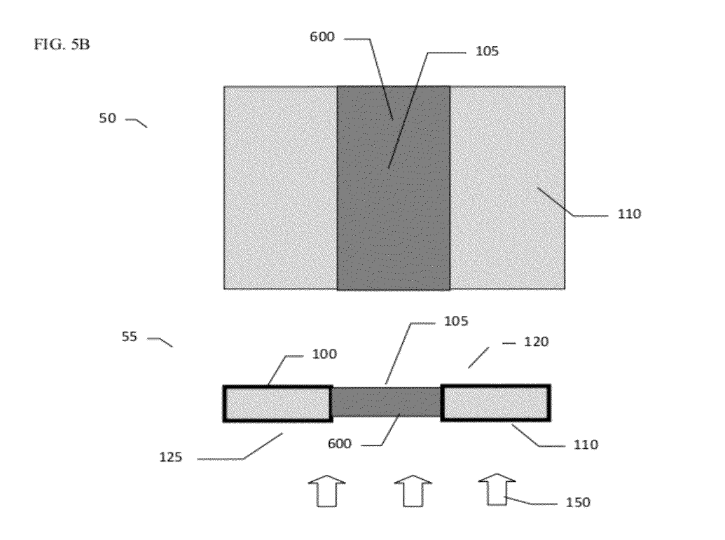

Referring now to FIGS. 5A and 5B, the thermal treatment device has a hotter center area as further illustrated by the denser shadowing of the center on (body-facing side) view 50. In the embodiments shown, a hotter heat mixture is filled into compartments in the middle of the thermal treatment device while a warm and/or cooler heating mixture is filled into compartments on the periphery.

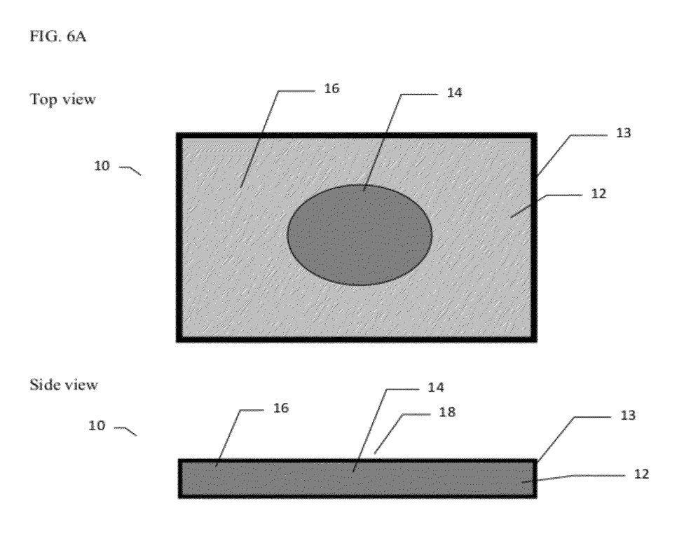

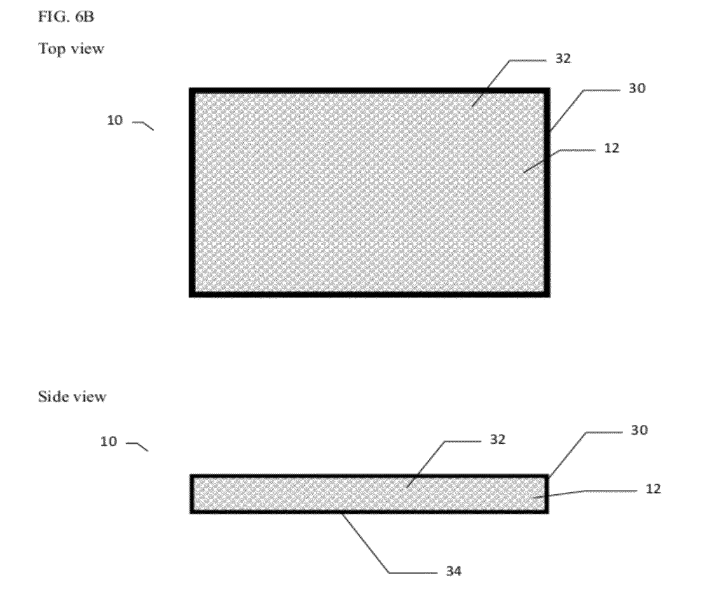

Referring to FIG. 6A, a thermal treatment device 10 in the form of a patch for providing therapy to an individual is shown. The thermal treatment device 10 may or may not be disposable, and includes a body 12 that applies therapy to the individual when the thermal treatment device 10 is placed on the individual. The body 12 includes and is enclosed by an outer surface 13. As used herein, patch refers to any type of patch, pack, bag, or pouch that may be used to apply therapy to a body. In addition, the thermal treatment device 10 may be capable of being attached directly, or indirectly, to an individual.

The thermal treatment device includes hot portions 14 and warm portions 16 that provide sufficient heat to make hot and warm, respectively, when applied to an individual's skin. In one aspect of the present disclosure, the thermal treatment device 10 is maintained in at least partial skin contact with a wearer of the thermal treatment device 10 due to the positioning of the thermal treatment device 10 on a skin-facing surface 18 of the body 12.

In one aspect of the present disclosure, hot and warm portions 14, 16 of the thermal treatment device 10 are formed by applying thermally insulating and/or thermally conductive materials to the skin-facing surface 18 of the thermal treatment device 10. Thermally insulating materials include any woven or non-woven fabric or material, for instance in the form of a pad, made of synthetic or natural polymer, or foam-like pad, for instance made of polyurethane. Thermally conductive materials include metal-based materials and/or composites, such as aluminum foil or fabric containing with metallic fibers or metal-filled polymer. In various aspects of the present disclosure, skin adhesive may be applied to the skin-facing surface 18. Skin adhesives may be comprised of multiple materials and bonding strengths, including but not limited to, soft skin adhesives, such as the 7-9700 grade commercially available from the Dow Corning corporation, spirit gums, silicone based adhesives, polyvinyl pyrrolidone and cross-linked polyvinylpyrrolidone based adhesives; and pressure sensitive adhesives, including those made from but not limited to, acrylic based polymers and copolymers, polyvinyl ethers, and silicones. The adhesive may be applied to the entire face of the thermal treatment device, or may be applied to only a portion of a face of the thermal treatment device. In one embodiment, the adhesive has a paper or film backing which is removed prior to application on the skin. The hot and warm sources may be applied in a variety of configurations. Different hot and/or warm portions 14, 16 may be colored using inks, dyes, or any other suitable substance to inform the user of the intended effect.

Referring to FIG. 6B, the body 12 of the thermal treatment device 10 may include an enclosure 30 and a heating composition 32 that is sealed inside the enclosure 30. The heating composition 32 is a heat generator and may be capable of generating heat when a gas, such as oxygen contained in ambient air, is received through a gas-permeable section 34 of the enclosure 30.

When the thermal treatment device 10 is a heat patch, it may be stored in a hermetic environment (e.g., a sealed bag) such that the heating composition remains inactive until the heat patch is removed from the hermetic environment. Once the thermal treatment device 10 is removed from the hermetic environment, the heating composition 32 within the enclosure 30 is exposed to air such that an exothermic reaction takes place within the body 12 of the thermal treatment device 10. The exothermic reaction generates heat within the thermal treatment device 10 to increase the temperature of the thermal treatment device 10. In one embodiment, the exothermic reaction in the hot portion of the thermal treatment device occurs at an increased rate compared to that in the warm portion of the thermal treatment device.

Any conventional heating composition may be used to induce an exothermic reaction within the thermal treatment device 10. Some example heating compositions include iron powder as the main active ingredient. Alternatively, the thermal treatment device 10 may include as a heat generator any suitable electrical heating system. Also alternatively, the thermal treatment device 10 may include as a heat generator any suitable fluid, gel, or solid heat storage systems that can be heated in a microwave oven, in a conventional oven, in a water bath, or by any other suitable means.

For the aspect in which at least one of the hot and/or warm portions 14, 16 relies on a chemical reaction, that portion of the thermal treatment device can be activated in a number of ways. The preferred way of activating is to expose the thermal treatment device to air.

For the aspect in which at least one of the hot and/or warm portions 14, 16 relies on an electrical or mechanical device, that portion of the thermal treatment device can be activated in a number of ways. The user may operate a switch to complete a circuit to supply electricity to that portion. The user may open valves leading to a source of hot or warm fluid.

According to the present invention, there is a thermal gradient along the heat pack surface at the interface with the user's body. In one embodiment, the center of the pack is heated more than the periphery of the pack, thus providing thermal therapy with thermal gradient. In another embodiment, the center of the pack is heated less (is cooler) vs. the periphery of the pack, thus avoiding overheating the area of the body immediately adjacent to the center of the pack.

Advantageously, the variable heat distribution (or thermal gradient) along the pack results in a the possibility of treating small areas of the body with higher levels of heat while treating surrounding areas of the body with more moderate levels of heat, thus avoiding overheating large areas while still providing substantial thermal treatment and relief. Further, for an affected area on the body being treated with heat, it is possible to heat the areas around, while avoiding directly heating of the affected area.

Heat packs according to this invention have appropriate heating mixture composition (typically iron, salts, water, carbon, filler), particle size (finer particles have higher surface area and result in higher temperature), and air permeability of the pouch, with high air permeability resulting in higher temperature. Packs are substantially flat packs with thickness of about 1 mm to about 30 mm, more preferably from about 3 mm to about 10 mm. The packs are of usual construction, comprising an air-permeable pouch filled with the air-activated heating mixture. The construction of the pack and the air-activated heating mixture can vary to result in higher or lower temperature: e.g., the pack could contain (i) specially formulated mixture (including different concentrations of components, or additives, such as salts or more active metals such as aluminum, and/or finer particle size of some of ingredients); (ii) higher air permeability of the pouch; or (iii) both (i) and (ii), to provide higher temperatures.

A number of embodiments of heat packs are described herein providing for the heat pack with variable heat distribution In the following figures, deeper grey color generally indicates higher temperature areas, with lighter grey color generally indicating lower temperature areas, and white color indicating even lower temperature areas, and/or less heated or non-heated areas.

In one embodiment, air ingress into a heat pack is variable along the pack. In a pack that is tightly packed and flat with substantially no movement of the heating mixture inside the bag or pouch (no clustering of the heating mixture, also known as the “tea-bagging” effect), the air permeability is made to be variable. For example, with higher air permeability on the periphery and lower in the middle, the periphery will heat up higher, and the middle will heat up lower. Higher air permeability in the middle will result in the middle of the pack being hotter. In another embodiment, a pack that is not tightly packed and allows for some movement of the heating mixture inside the bag or pouch (“tea-bagging” effect), the air permeability is similarly made to be variable. For example, with higher air permeability on the periphery and lower in the middle, the periphery will heat up higher, and the middle will heat up lower. Higher air permeability in the middle will result in the middle of the pack being hotter.

Referring again to FIGS. 1A, 1B and 1C-1 to 1C-6, which show a thermal treatment device of the present invention, wherein (a) the device has a hot center and a warm periphery (FIG. 1A); (b) the device has a warm center and a hot periphery (FIG. 1B); and (c) the device has a non-heated center and a hot periphery (FIGS. 1C-1 to 1C-6).

Air permeability can be varied by using a specialized fabric or by having more apertures or additional apertures or apertures of larger diameter made in the area where higher permeability is desired. In one embodiment, additional needle-formed micro-apertures are made in the pouch in the area where higher temperature is desired. Referring again to FIGS. 2A-1 to 2A-2 and 2B, which show a thermal treatment device of the present invention, wherein (a) the device has a hot center with apertures and a warm periphery (FIGS. 2A-1 to 2A-2); and (b) the device has a warm center and a hot periphery with apertures (FIG. 2B).

In one embodiment, the thermal gradient is achieved by variability of insulation of the heat pack at the interface with the user's body. The insulation is thicker fabric or pressure-sensitive tape on the surface of the pack facing the body. Referring again to FIGS. 3A, 3B, and 3C-1 to 3C-2, which show a thermal treatment device of the present invention, wherein (a) the device has a non-insulated hot center and an insulated warm periphery (FIG. 3A); (b) the device has an insulated warm center and a non-insulated hot periphery (FIG. 3B); and (c) the device has concentric insulation around a hot center (FIG. 3C-1 to 3C-2).

In one embodiment, the hot and warm portions and thus the hot and warm heat delivery may be achieved through the use of different apertures in each portion. In one embodiment, the hot portion may have more apertures than the warm portion.

The casing of the thermal treatment device, or the sub-packs or heating composition may be comprised of various woven or non-woven fabric materials. In one embodiment, the casing of the hot portion has a higher porosity than the casing of the warm portion. The thermal treatment device may be constructed of different shapes as discussed above, but the sub-pack portions of the heating device may also be comprised of different shapes, including shapes which are stitched or sealed into the total thermal treatment device. These stitched or sealed portions do not contain the internal heating composition. In one embodiment, the shape of the hot portion is the same as the shape of the warm portion. In one embodiment, the shape of the hot portion is different than the shape of the warm portion.

In another embodiment the heating pack comprises at least two sub-packs, with one sub-pack providing more heat vs. another. Referring again to FIGS. 4A and 4B, which show a thermal treatment device of the present invention, wherein (a) the device has a hot center and a warm periphery prepared by placing a hot sub-pack on top of a warm sub-pack (FIG. 4A); and (b) the device has a warm center and a hot periphery prepared by placing a warm sub-pack between two hot sub-packs (FIG. 4B). The sub-packs may be attached to one another through various means. In one embodiment, the sub-pack is attached to another sub-pack by an adhesive. In another embodiment, where the sub-packs are surrounded by a casing, the sub-pack and its heating composition are portioned from other sub-packs through quilting, sealing or stitching. In one embodiment, the hot sub-pack may be removed from the larger thermal treatment device after the user has determined that the sub-pack is extinguished. This removal may be facilitated by a pressure sensitive adhesive or by a perforated portion that can be detached. In one embodiment, the hot sub-pack portion has an indicator which allows the user to know that it has been extinguished, and can be removed or that the thermal treatment device position can be changed. This indicator may be an electronic indicator such as an led or a color indicator which appears as a separate color when the hot portion is extinguished. In one embodiment, there may be more than one hot sub-pack attached to one warm sub-pack. In one embodiment, there may be more than one warm sub-pack.

In another embodiment, heat pack has at least two compartments filled with different air-activated heating mixtures or having different air permeability, or both. Referring to FIGS. 5A and 5B, which show a thermal treatment device of the present invention, wherein (a) the device has a hot center and a warm periphery, wherein two or more compartments are filled with different air-activated heat mixtures; and (b) the device has a hot center and a warm periphery, wherein three or more compartments are filled with different air-activated heat mixtures. In one embodiment, there may be a portion that is stitched or sealed between the hot portion center and the warm portion periphery which does not comprise the heating composition. In one embodiment, the hot sub-pack is placed on top of the warm sub-pack wherein the warm heating composition comprised the entire surface area of the thermal treatment device and transfers heat through the hot portion, and can be felt when the hot portion is extinguished.

The packs can be rectangular, circular, polygonal, or of any geometric shape suitable for placing on the body or around the body. The packs can be worn on the body via attachment with adhesive to the skin or to the clothing, by being inserted into a belt, sleeve, or wrap, or by securing with straps, having Velcro attachments, adhesive, or other means to attach to the body, clothing, or to another strap.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

1. A thermal treatment device comprising:
an outer surface comprising a skin facing surface, wherein said skin facing surface comprises variable heat distribution.

2. The thermal treatment device of claim 1, wherein the skin facing surface has a hot center and a warm periphery.

3. The thermal treatment device of claim 1, wherein the skin facing surface has a warm center and a hot periphery.

4. The thermal treatment device of claim 1, wherein the skin facing surface has a non-heated center and a hot periphery.

5. The thermal treatment device of claim 1, wherein the skin facing surface has a hot center with air-permeation apertures and a warm periphery.

6. The thermal treatment device of claim 1, wherein the skin facing surface has a warm center and a hot periphery with air-permeation apertures.

7. The thermal treatment device of claim 1, wherein the skin facing surface has a non-insulated hot center and an insulated warm periphery.

8. The thermal treatment device of claim 1, wherein the skin facing surface has an insulated warm center and a non-insulated hot periphery.

9. The thermal treatment device of claim 1, wherein the skin facing surface has concentric insulation around a hot center.

10. The thermal treatment device of claim 1, wherein the skin facing surface has a hot center and a warm periphery prepared by placing a hot sub-pack on top of a warm sub-pack.

11. The thermal treatment device of claim 1, wherein the skin facing surface has a warm center and a hot periphery prepared by placing a warm sub-pack between two hot sub-packs.

12. The thermal treatment device of claim 1, wherein the skin facing surface has a hot center and a warm periphery, wherein two or more compartments are filled with different air-activated heat mixtures.

13. The thermal treatment device wherein the skin facing surface has a hot center and a warm periphery, wherein three or more compartments are filled with different air-activated heat mixtures.

14. The thermal treatment device of claim 1, further comprising a thermal composition.

15. The thermal treatment device of claim 14, wherein the thermal composition emits heat from about 1 to about 10° C. above the skin surface temperature of an individual when worn next to the skin of said individual.

16. The thermal treatment device of claim 14, wherein the thermal composition comprises iron powder.

17. The thermal treatment device of claim 14, wherein the thermal composition comprises a microwavable heat retaining material.

18. A thermal treatment device, comprising:
a skin-facing surface;
at least one hot portion disposed on the skin-facing surface; and
at least one warm portion disposed on the skin-facing surface.

19. A method of treatment comprising:
exposing an individual's skin to a thermal treatment device, comprising:
a skin-facing surface;
at least one hot portion disposed on the skin-facing surface; and
at least one warm portion disposed on the skin-facing surface.

20. The thermal treatment device of claim 1, further comprising a surface opposite the skin facing surface, wherein the surface opposite the skin facing surface has variable air permeability.

21. The thermal treatment device of claim 20, wherein the surface opposite the skin facing surface has air permeation apertures, said air permeation apertures having size and or density which are different in a center of said surface opposite the skin facing surface versus in a periphery of said surface opposite the skin facing surface.

22. The thermal treatment device of claim 1, further comprising at least one cut-out in the skin facing surface.
